(19) United States
(12) Patent Application Publication
(10) Pub. No.: US 2018/0153744 A1
(43) Pub. Date: Jun. 7, 2018

(54) INJECTION SITE MULTI-FUNCTIONAL BADAGE

(71) Applicant: ABDULRAHMAN S.J.M. AL-HERAIBI, SAFAT (KW)

(72) Inventor: ABDULRAHMAN S.J.M. AL-HERAIBI, SAFAT (KW)

(21) Appl. No.: 15/825,943

(22) Filed: Nov. 29, 2017

Related U.S. Application Data
(60) Provisional application No. 62/430,362, filed on Dec. 6, 2016.

(51) Int. Cl. A61F 13/02; A61F 13/00

(52) U.S. Cl. CPC A61F 13/023; A61F 2013/0017; A61F 13/00063; A61F 13/0206

(57) ABSTRACT

The injection site multi-functional bandage includes a thin, flexible strip of support material with opposing upper and lower surfaces. The support material can be elongated with adhesive portions at opposing ends thereof. The adhesive portions can be covered by a removable cover. A non-adhesive absorbent can be provided between the adhesive portions on the lower surface of the support material. A reservoir including sterilization liquid can be disposed above or below the absorbent. The reservoir can be positioned along a center portion of the support material. An opening can extend through a portion of the absorbent. The opening can be selectively sealable by a releasable flap.

### CROSS-REFERENCE TO RELATED APPLICATION

This application claims the benefit of U.S. Provisional Patent Application Ser. No. 62/430,362, filed Dec. 6, 2016.

### 1. FIELD

The injection site multi-functional bandage relates generally to medical devices. More specifically, the injection site multi-functional bandage is a bandage capable of sterilizing an injection site, providing access to an injection site, and then re-sealing to protect the injection site.

### 2. DESCRIPTION OF THE RELATED ART

The risk to health workers from contracting contagious blood related diseases, such as hepatitis, has always been a consideration but increased concern has undoubtedly arisen since the more recent advent of the HIV virus.

Despite these risks, most research into protecting health workers has centered on the safer disposal of blood contaminated products (such as syringe needles). However, very little, if anything, has been done to protect the health worker during procedures such as the giving of an injection. In fact, the basic procedure remains substantially unchanged. This is typically to inject the patient with a needle, withdraw the needle and then apply a dressing to absorb any blood from the wound or withdrawn with the needle. It is very easy and common for blood to spill on the patient's skin which then becomes a potential source of infection. This then gives rise to the possibility of the person giving the injection contracting a blood born infection.

Injections and the insertion of cannulas are not the only operations to be performed which may place users at risk, or where improvements may be made. For instance, minor operations such as the lancing of boils, removal of moles and cancerous growths, and closing minor incisions, are generally performed before a dressing is applied. In some instances, it is desirable to clean around the wound or afflicted area before applying a dressing, especially dressings of an adhesive type which must maintain good contact with the areas surrounding the wound or afflicted site. In many instances, it would be useful to use a dressing which surrounds a wound or site before any minor operation is performed, and which could be readily cleaned or wiped, and closed.

It is an object of the present invention to address the foregoing problems or at least to provide the public with a useful choice. Further aspects and advantages of the present invention will become apparent from the ensuing description which is given by way of example only.

### SUMMARY

An injection site multi-functional bandage includes a thin, flexible strip of support material with opposing upper and lower surfaces. The support material can be elongated with adhesive portions at opposing ends thereof. The adhesive portions can be covered by a removable cover. A non-adhesive absorbent can be provided between the adhesive portions on the lower surface of the support material. A reservoir including sterilization liquid can be disposed above or below the absorbent. The reservoir can be positioned along a center portion of the support material. An opening can extend through a portion of the absorbent. A releasable flap on the support material can be selectively lifted to provide access to the opening.

These and other features of the present invention will become readily apparent upon further review of the following specification and drawings.

### BRIEF DESCRIPTION OF THE DRAWINGS

FIG. 1 is a perspective view of an upper surface of an injection site multi-functional bandage.

FIG. 2 is an environmental view illustrating the process for using the injection site multi-functional bandage.

FIG. 3 is a perspective view of the lower surface of an injection site multi-functional bandage with the cover.

FIG. 4 is a perspective view of the lower surface of an injection site multi-functional bandage without the cover

Similar reference characters denote corresponding features consistently throughout the attached drawings.

### DETAILED DESCRIPTION OF THE PREFERRED EMBODIMENTS

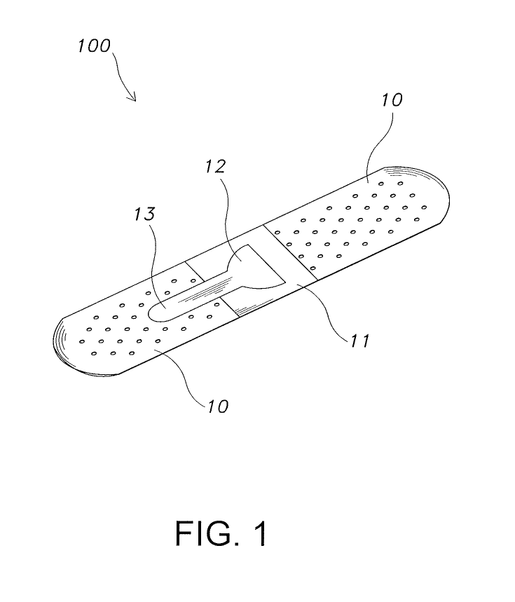

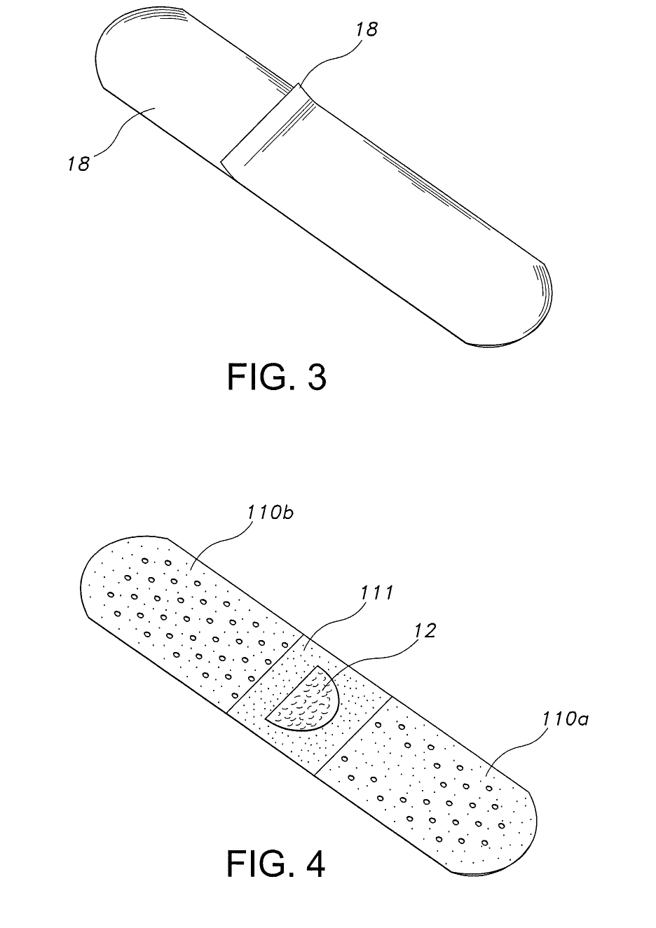

The injection site multi-functional bandage, designated 100 in FIGS. 1-3, relates generally to medical devices. More specifically, the injection site multi-functional bandage 100 can include a thin, flexible strip of support material having opposing upper and lower surfaces. The support material 10 can be a woven or non-woven material suitable for binding or covering a wound. The support material 10 can be elongated with adhesive portions 110a, 110b at opposing ends thereof. The adhesive portions 110a, 110b can be covered by a removable cover 18. A non-adhesive absorbent 111, such as a cotton pad, can be provided between the adhesive portions 110a, 110b at the lower surface of the support material 10.

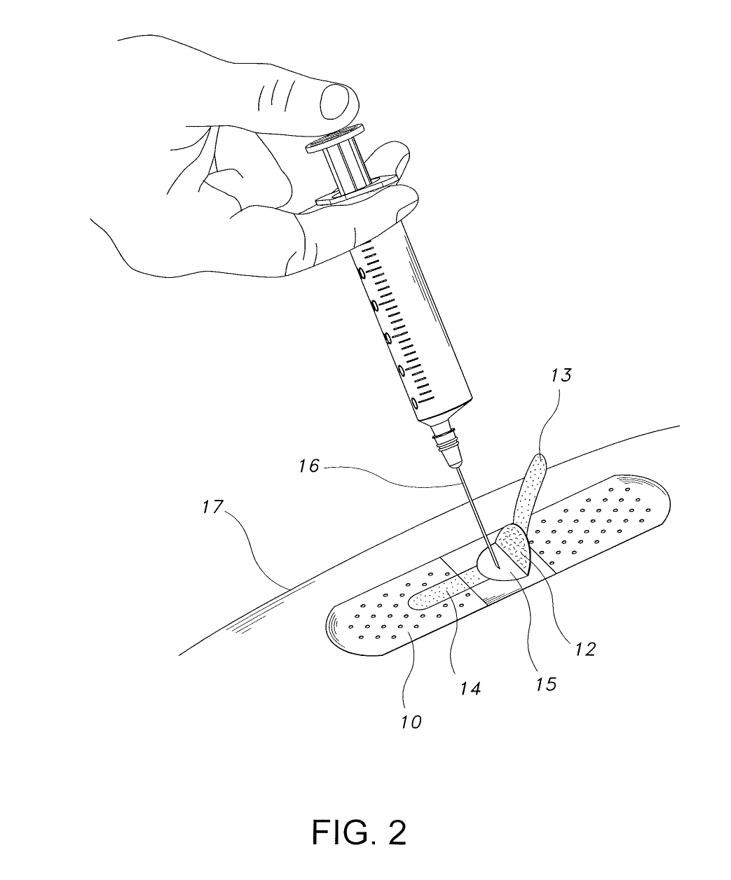

A reservoir 12 including sterilization liquid, can be disposed above or below the absorbent 111. The reservoir 12 can be positioned along a center portion of the support material 10. An opening 15 can extend through a portion of the absorbent 111. The opening 15 can be selectively sealable by a releasable flap 13. A first portion of the flap 13 can include an adhesive to facilitate releasable attachment to an upper surface of the elongated strip, e.g., to one end thereof. A second portion of the flap 13 may include an absorbent that is configured to fit within the opening 15. When the flap 13 is opened, the patient's skin is exposed through the opening 15. As shown in FIG. 2, once the flap 13 is opened, an injection can be delivered through the opening 15.

In use, the cover 18 is removed and the bandage 100 is applied to a skin surface where an injection is to be delivered. Pressure is applied to the reservoir 12 to release the sterilization liquid. The flap 13 is then opened to provide access to the opening 15. An injection needle 16 is inserted through the opening 15 and into the patient's skin. The flap 13 may then be closed to protect the injection site.

It is to be understood that the injection site multi-functional bandage is not limited to the embodiments described above, but encompasses any and all embodiments within the scope of the following claims.

I claim:

1. An injection site multi-functional bandage, comprising:
a flexible support material;
first and second adhesive portions on a lower surface of the support material;
a non-adhesive absorbent between the first and second adhesive portions at the lower surface of the support material, the non-adhesive absorbent including an aperture defined therethrough;
a releasable flap disposed over the aperture for selectively sealing the aperture; and
a cover releasably attached to the first and second adhesive portions.

2. The injection site multi-functional bandage of claim 1, further comprising a reservoir below the releasable flap, the reservoir including a sterilization liquid.

3. The injection site multi-functional bandage of claim 2, wherein the reservoir is disposed between the flap and the absorbent.

4. The injection site multi-functional bandage of claim 1, wherein the flexible support material is a woven material.

5. The injection site multi-functional bandage of claim 1, wherein the flexible support material is a non-woven material.

6. The injection site multi-functional bandage of claim 1, wherein the non-adhesive absorbent includes cotton.

7. The injection site multi-functional bandage of claim 1, wherein the releasable flab includes a non-adhesive absorbent on a first portion thereof and an adhesive on a second portion thereof.

8. A method for using an injection site multi-functional bandage, comprising:
providing an injection site multi-functional bandage including
a flexible support material;
first and second adhesive portions on a lower surface of the support material;
a non-adhesive absorbent between the first and second adhesive portions on the lower surface, the non-adhesive absorbent including an aperture defined therethrough;
a releasable flap on an upper surface of the support material, the releasable flap selectively sealing the aperture;
a reservoir including sterilization liquid along a central portion of the support material, and
a cover releasably attached to the first and second adhesive portions;
removing the releasable cover from the lower surface of the bandage;
positioning the bandage on a skin surface injection site;
opening the flap to provide access to the skin surface injection site;
delivering an injection to the skin surface through the opening; and
closing the flap to cover the opening and protect the injection site.

9. The method for using an injection site multi-functional bandage according to claim 8, further comprising the step of:
applying pressure to the reservoir to release the sterilization fluid prior to opening the flap.
